(19) United States
(12) Patent Application Publication
(10) Pub. No.: US 2006/0084603 A1
(43) Pub. Date: Apr. 20, 2006

(54) IGF-BP3-related methods for inhibiting tumor growth

(76) Inventors: Irena Kirman, Briarwood, NY (US); Richard Whelan, New York, NY (US)

Correspondence Address: COOPER & DUNHAM, LLP, 1185 AVENUE OF THE AMERICAS, NEW YORK, NY, 10036, US

(21) Appl. No.: 11/197,874

(22) Filed: Aug. 5, 2005

Related U.S. Application Data
(60) Provisional application No. 60/599,380, filed on Aug. 6, 2004.

(51) Int. Cl. A61K 38/18

(52) U.S. Cl. 514/12

(57) ABSTRACT

The subject invention provides a method of inhibiting the proliferation of cells associated with a tumor in a subject which comprises administering to the subject between about 10 μg/kg and about 100 mg/kg of IGF-BP3, thereby inhibiting proliferation of the cells. The subject invention further provides a surgical method which comprises surgically resecting a tumor from a subject and administering to the subject between about 10 μg/kg and about 100 mg/kg of IGF-BP3, so as to inhibit metastasis of any tumor cells released in the subject's blood circulation during resection of the tumor. The subject invention further provides a surgical method which comprises performing a surgical procedure on a subject and administering to the subject between about 10 μg/kg and about 100 mg/kg of IGF-BP3, so as to inhibit proliferation of a tumor cell in the subject. The subject invention also provides an article of manufacture comprising a packaging material having therein IGF-BP3 in an amount suitable for administering a dosage to a subject of between about 10 μg/kg and about 100 mg/kg, and instructions for using the IGF-BP3 prior to, during and/or after a surgical procedure performed on the subject.

This application claims the benefit of U.S. Provisional Application No. 60/599,380, filed Aug. 6, 2004, the contents of which are incorporated herein by reference into the subject application.

Throughout this application, various publications are referenced by the first author's last name or by Arabic number in parenthesis. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

The mainstay of treatment for the vast majority of intestinal and visceral malignancies has been “radical” resection of the tumor via laparotomy. In the past decade an alternative abdominal access method, namely laparoscopy, has been utilized, by some, for the curative resection of malignancies. This use of minimally invasive methods remains controversial because of the lack of long-term studies and concerns about port wound tumors. Early results from randomized trials comparing traditional to laparoscopic-assisted colon resection for cancer have shown that an adequate minimally invasive oncologic resection can be done (Milsom, J. W., et al.)

Numerous experimental studies have demonstrated that laparotomy, when compared to CO₂ pneumoperitoneum or anesthesia alone, is associated with increased rates of tumor establishment and growth (Shiromizu, A., et al.; Allendorf, J. D., et al., 1995; Southall, J. C, et al.; Lee, S. W., et al., 1999). Similar results were noted after open, and closed bowel resection (Allendorf, J. D., et al., 1998). Tumor cell proliferation was shown to be increased and apoptosis decreased after laparotomy in a murine study (Lee, S. W., et al., 1998). The mechanism of these tumor growth differences has also been investigated. Laparotomy related inhibition of immune function may account for some of the observed differences in tumor growth after surgery (Allendorf, J. D., et al., 1999; Da Costa, M. L., et al.). Laparotomy associated elevation of circulating active protein substances, such as VEGF may also play a role (Pidgeon, G. P., et al. ). In an animal study that assessed the ability of pre- and postoperative mouse plasma to support tumor cells in vitro, significantly greater growth was noted in cultures to which post laparotomy serum (from postoperative days 2 and 4) had been added (Lee, S. W., et al., 2000). It was postulated that a surgery-related plasma factor accounted for the differences observed. In another study done with the same model, the factor was identified as platelet derived growth factor (PDGF) (Lee, S. W., et al., 2001).

### SUMMARY OF THE INVENTION

The subject invention provides a method of inhibiting the proliferation of cells associated with a tumor in a subject which comprises administering to the subject between about 10 μg/kg and about 100 mg/kg of IGF-BP3, thereby inhibiting proliferation of the cells.

The subject invention further provides a surgical method which comprises surgically resecting a tumor from a subject and administering to the subject between about 10 μg/kg and about 100 mg/kg of IGF-BP3, so as to inhibit metastasis of any tumor cells released in the subject's blood circulation during resection of the tumor.

The subject invention further provides a surgical method which comprises performing a surgical procedure on a subject and administering to the subject between about 10 μg/kg and about 100 mg/kg of IGF-BP3, so as to inhibit proliferation of a tumor cell in the subject.

The subject invention also provides an article of manufacture comprising a packaging material having therein IGF-BP3 in an amount suitable for administering a dosage to a subject of between about 10 μg/kg and about 100 mg/kg, and instructions for using the IGF-BP3 prior to, during and/or after a surgical procedure performed on the subject.

### DESCRIPTION OF THE FIGURES

FIG. 1. Correlation between the Increase in OS Plasma Mitogenic Activity on POD1 and the Length of Incision. HT29 cells were incubated with 10% plasma from patients undergoing open surgery and BrdU incorporation test performed. A percentage increase in BrdU+ cells on POD1 versus preOP was calculated and plotted to the length of the incision.

FIG. 2. IGF-BP3 Western Blots. Each pair of lanes is one patient's PreOP and POD1 results; Lanes 1 & 2 show an OS patient; Lanes 3 & 4 and 5 & 6, respectively, are 2 LS patients' results. IGF-BP3 was notably decreased in the OS patient on POD1 (Lane 2) versus PreOP (Lane 1) , but not in the LS patients (Lanes 4 and 6 vs. Lanes 3 and 5).

FIG. 3. Direct Inhibitory Effect of IGF-BP3 on Growth of Colon Cancer Cells. HT29 cells were plated in serum free conditions with IGF-BP3 in various concentrations. The resulting number of recovered cells (left) and the percentage of BrdU+ cells in cultures decreased with increasing concentrations of IGF-BP3.

FIG. 4. Neutralization of the Mitogenic Effect. Each triplet displays one patient's results (unshaded bars, PreOP results; black bars, POD1 results; crosshatched bars, rhIGFBP3 supplemented POD1 results). Recombinant human IGF-BP3 was added to cell cultures containing 10% POD1 OS plasma. The percentage of BrdU+ cells and the total cell count were decreased in supplemented wells (p<0.05 vs. PreOp Plasma) compared to results with POD1 plasma alone (closed). RhIGF-BP3 POD1 OS plasma vs. PreOP OS plasma, no difference noted.

FIG. 5. Impact of Anti-IGFBP3 Antibody on the Mitogenic Effect of PreOP OS Plasma. Each triplet displays one patient's results (unshaded bars, PreOP results; crosshatched bars, ab+PreOp Plasma results; black bars, POD1 OS results). Neutralizing antibody to IGF-BP3 was added to wells containing PreOP OS plasma (concentration per well 10 μg/ml). HT29 proliferation (counts, and BrdU incorporation) was significantly higher in antibody supplemented wells when compared to PreOP OS plasma results (p<0.05). The addition of antibody raised PreOp Plasma associated HT29 proliferation to levels observed with the POD1 OS plasma.

FIG. 6. The Effect of rhIGFBP-3 on Growth of CT26 Murine Colon Adenocarcinoma Cells. CT26 cells were plated 8×10⁵/well and allowed to grow 48 hours in serum free conditions with or without the addition of rhIGFBP-3. The difference in the number between the recovered cells from the control wells (open) and rhIGFBP-3 containing wells (filled) was statistically significant (p<0,01).

FIG. 7. rhIGFBP-3 Proteolysis Induced by CT26 Cells. CT26 cells were incubated with E.coli expressed bioactive fragment of rhIGFBP-3. Subsequently, IGFBP-3 was analyzed in supernatants by Western Blot analysis as described in Materials and Methods. Lane A, control CT26 cells, no rhIGFBP-3 added; Lane B, rhIGFBP-3 fragmentation after incubation with CT26 cells; Lane C, rhIGFBP-3 incubated in control cell free wells.

FIG. 8. The in vivo Effect of rhIGFBP-3 on Growth of CT26 Tumors. CT26 cells were inoculated in BALB/c mice, 10⁵/anima]. Test group received rhIGFBP-3, 50 μg concomitantly with inoculum and subsequently once a week, 100 μg of rhIGFBP-3, 2 times. Tumors were allowed to grow 2.5 weeks. The difference in tumor weight between the rhIGFBP-3 treated and control groups was statistically significant (p<0.01).

FIG. 9. Origin of Cells in the CT26 Tumor Mass. Tumors were excised and a single cell suspension prepared. Cells were then ethanol fixed, washed and stained with an antibody to pancytokeratin-FITC and analyzed by flow cytometry. Shown is the representative histogram of cytokeratin expression, solid line and of an isotype matched control, dotted line.

FIG. 10. The Number of Aberrant Crypt Foci Induced by AOM Treatment in IGFBP3-TG and WT mice. IGFBP3-TG and WT mice were treated with AOM as described in Materials and Methods. At the end of the experiment, mice were sacrificed, their colons removed, opened, fixed and stained with methylene blue as described in Materials and Methods and the number of ACF/colon counted under the inverted microscope. The difference in the number of ACF/colon between IGFBP3-TG and WT mice was statistically significant (p<0.001).

FIG. 11. Concentration of Total IGFBP-3 in Colon Cancer Patients undergoing Open or Laparoscopic Assisted Surgery. Total IGFBP-3 was assayed in EDTA plasma using ELISA as described in Materials and Methods. *The mean total IGFBP-3 concentration value was significantly lower on POD2 than before surgery in OS (p<0.05) but not in LS group.

FIG. 12. Representative Western Blot Analysis of IGFBP-3 in Plasma from Colon Cancer Patients Undergoing Open or Laparoscopic Assisted Surgery. Assays were performed using EDTA plasma and immunomagnetic separation of products from the following samples. Open surgery: patient A preOP (lane 1) and POD2 (lane 2); patient B preOP (lane 3), POD2 (lane 4) and POD3 (lane 5); patient C preOP (lane 6), POD1 (lane 7) and POD2 (lane 8), patient D preOP (lane 9), POD1 (lane 10) and POD2 (lane 11). Laparoscopic surgery: patient E preOP (lane 12) and POD2 (lane 13); patient F preOP (lane 14) and POD2 (lane 15); patient G preOP (lane 16), POD1 (lane 17) and POD2 (lane 18).

FIG. 13. Concentration of Intact IGFBP-3 in Colon Cancer Patients undergoing Open or Laparoscopic Assisted Surgery. Intact IGFBP-3 was assessed in EDTA plasma samples using a combined ELISA and Western Blot assay as described in Materials and Methods. ***In OS patients, the mean intact IGFBP-3 concentration value was statistically lower in POD2 than in preOP samples (p<0.0003) and than in POD2 samples from LS patients (p<0.03).

### DETAILED DESCRIPTION OF THE INVENTION

This invention provides a method for inhibiting the proliferation of cells associated with a tumor in a subject which comprises administering to the subject between about 10 μg/kg and about 100 mg/kg of IGF-BP3, thereby inhibiting proliferation of the cells. In one embodiment, between about 100 μg/kg and about 10 mg/kg of IGF-BP3 is administered to the subject. In another embodiment, between about 500 μg/kg and about 5 mg/kg of IGF-BP3 is administered to the subject. In another embodiment, about 0.7 mg/kg, 0.8 mg/kg, 0.9 mg/kg, 1.0 mg/kg, 1.1 mg/kg, 1.2 mg/kg, 1.3 mg/kg, 1.4 mg/kg or 1.5 mg/kg is administered to the subject. In another embodiment, the subject is human. In another embodiment, the administration is intravenous. In another embodiment, the administration is repeated weekly for up to six weeks. In another embodiment, the tumor is associated with colon cancer, prostate cancer, breast cancer or lung cancer. In another embodiment, the tumor is associated with colon cancer. In another embodiment, the IGF-BP3 is recombinantly produced IGF-BP3. In another embodiment, the IGF-BP3 is human IGF-BP3. In another embodiment, the administering is performed prior to, during and/or after surgery. In another embodiment, the surgery is open abdominal surgery. In another embodiment, the surgery is laparoscopic surgery. In another embodiment, the subject, prior to IGF-BP3 administration, has been determined to have a low blood level of IGF-BP3.

This invention further provides a surgical method which comprises surgically resecting a tumor from a subject and administering to the subject between about 10 μg/kg and about 100 mg/kg of IGF-BP3, so as to inhibit metastasis of any tumor cells released in the subject's blood circulation during the surgical resection of the tumor. In one embodiment, between about 100 μg/kg and about 10 mg/kg of IGF-BP3 is administered to the subject. In another embodiment, between about 500 μg/kg and about 5 mg/kg of IGF-BP3 is administered to the subject. In another embodiment, about 0.7 mg/kg, 0.8 mg/kg, 0.9 mg/kg, 1.0 mg/kg, 1.1 mg/kg, 1.2 mg/kg, 1.3 mg/kg, 1.4 mg/kg or 1.5 mg/kg is administered to the subject. In another embodiment, the subject is human. In another embodiment, the administration is intravenous. In another embodiment, the administration is repeated weekly for up to six weeks. In another embodiment, the tumor is associated with colon cancer, prostate cancer, breast cancer or lung cancer. In another embodiment, the tumor is associated with colon cancer. In another embodiment, the IGF-BP3 is recombinantly produced IGF-BP3. In another embodiment, the IGF-BP3 is human IGF-BP3. In another embodiment, the administering is performed prior to, during and/or after surgery. In another embodiment, the administering is performed during surgery. In another embodiment, the administering is performed after surgery. In another embodiment, the administering is performed during and after surgery. In another embodiment, the surgery is open abdominal surgery. In another embodiment, the surgery is laparoscopic surgery. In another embodiment, the subject, prior to IGF-BP3 administration, has been determined to have a low blood level of IGF-BP3.

This invention further provides a surgical method which comprises performing a surgical procedure on a subject and administering to the subject between about 10 μg/kg and about 100 mg/kg of IGF-BP3, so as to inhibit proliferation of a tumor cell in the subject. In one embodiment, between about 100 μg/kg and about 10 mg/kg of IGF-BP3 is administered to the subject. In another embodiment, between about 100 μg/kg and about 5 mg/kg of IGF-BP3 is administered to the subject. In another embodiment, about 0.7 mg/kg, 0.8 mg/kg, 0.9 mg/kg, 1.0 mg/kg, 1.1 mg/kg, 1.2 mg/kg, 1.3 mg/kg, 1.4 mg/kg or 1.5 mg/kg is administered to the subject. In another embodiment, the subject is human. In another embodiment, the administration is intravenous. In another embodiment, the administration is repeated weekly for up to six weeks. In another embodiment, the tumor is associated with colon cancer, prostate cancer, breast cancer or lung cancer. In another embodiment, the tumor is associated with colon cancer. In another embodiment, the IGF-BP3 is recombinantly produced IGF-BP3. In another embodiment, the IGF-BP3 is human IGF-BP3. In another embodiment, the administering is performed prior to, during and/or after surgery. In another embodiment, the administering is performed during surgery. In another embodiment, the administering is performed after surgery. In another embodiment, the administering is performed during and after surgery. In another embodiment, the surgery is open abdominal surgery. In another embodiment, the surgery is laparoscopic surgery. In another embodiment, the subject, prior to IGF-BP3 administration, has been determined to have a low blood level of IGF-BP3.

Finally, this invention provides an article of manufacture comprising a packaging material having therein IGF-BP3 in an amount suitable for administering a dosage to a subject of between about 10 μg/kg and about 100 mg/kg, and instructions for using the IGF-BP3 prior to, during and/or after a surgical procedure performed on the subject. In one embodiment, the IGF-BP3 is in an amount suitable for administering a dosage to the subject of between about 100 μg/kg and about 10 mg/kg. In another embodiment, the IGF-BP3 is in an amount suitable for administering a dosage to the subject of between about 500 μg/kg and about 5 mg/kg. In another embodiment, the IGF-BP3 is in an amount suitable for administering a dosage to the subject of about 0.7 mg/kg, 0.8 mg/kg, 0.9 mg/kg, 1.0 mg/kg, 1.1 mg/kg, 1.2 mg/kg, 1.3 mg/kg, 1.4 mg/kg or 1.5 mg/kg. In another embodiment, the subject is human. In another embodiment, the administering is intravenous. In another embodiment, the administering is repeated weekly for up to six weeks. In another embodiment, the IGF-BP3 is recombinantly produced IGF-BP3. In another embodiment, the IGF-BP3 is human IGF-BP3.

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA). Agents administered according to this invention are preferably admixed with a pharmaceutically acceptable carrier.

“Subject” shall mean any organism including, without limitation, a mammal such as a mouse, a rat, a dog, a guinea pig, a ferret, a rabbit and a primate. In the preferred embodiment, the subject is a human being.

This invention will be better understood from the Experimental Details which follow. However, one skilled in the art will readily appreciate that the specific method and results discussed are merely illustrative of the invention as described more fully in the claims which follow thereafter.

### Experimental Details Part 1

### Materials

### Patients

Eighty-four patients (43 males, and 42 females) that underwent either a colorectal resection or a gastric bypass for morbid obesity were included in this study. Forty-five patients underwent open surgery, whereas 39 had minimally invasive procedures. Patients on corticosteroids, other immunosuppressive drugs, and those who had undergone chemotherapy or radiotherapy within 3 months of surgery were excluded from the study. Anesthesia was induced and maintained with propofol, succinyl choline and nitrous oxide. Additionally, fentanyl and magnesium sulfate were given. The study was approved by the institutional IRB and informed consent was obtained from all patients.

TABLE 1
Indications for Surgery

| Indication | OS group, n(%) | LS group, n(%) |
|---|---|---|
| Colorectal cancer | 20(44) | 22(56) |
| Morbid Obesity | 13(29) | 8(21) |
| Colorectal Adenoma | 8(18) | 5(13) |
| Diverticular | 4(9) | 4(10) |
| Disease | | |

OS denotes Open Surgery;
LS denotes Laparoscopic Surgery

TABLE 2
Accompanying Diseases in LS and OS Patients

| Accompanying Diseases | OS Group, n(%) | LS Group, n(%) |
|---|---|---|
| Coronary artery disease, | 14(31) | 16(41) |
| Hypertension | | |
| Chronic Obstructive | 5(11) | 3(8) |
| Pulmonary Disease | | |
| Asthma | | |
| Diabetes | 4(9) | 2(5) |
| Past surgery | 5(11) | 4(10) |

The indications for surgery are provided in Table 1 whereas Table 2 concerns associated illnesses. The two groups were statistically similar in regards to indication and associated illnesses, age (overall groups and subgroups), and mean height and weight for each group. Furthermore, in regards to the colon cancer patients, there were no significant differences noted in final tumor stage, size of tumor, overall length of specimen, number of lymph nodes, or margins. There were no conversions in the laparoscopic group. None of the patients in either group received perioperative blood transfusions. The mean length of incision was 19.4±4.7 cm in the OS group and 5.0±2.1 cm in LS group.

### Peripheral Blood Collection

Samples were collected in EDTA containing tubes pre-operatively and on post-operative days 1 and 3. Plasma was isolated by centrifugation soon after being drawn and stored at −80° C. until used.

### Tumor Cell Line

The HT29 tumor, a human colonic adenocarcinoma cell line, was obtained from ATCC (Manassas, Va.) and maintained in complete DMEM medium (Cellgro, Herndon, Va.) with 10% fetal calf serum (FCS) (Cellgro). For the assay, HT29 cells were plated in 6-well plates, 2×10⁵ cells/well in 2 ml of complete medium with FCS, and allowed to adhere. Cells were then washed 2 times with serum free DMEM medium and incubated for 48 hours with 10% human serum from the patients.

### Biological Testing

### 5-Bromo-deoxyuridine (BrdU) Incorporation Assay

Two hours before the end of the incubation period, cells were pulsed with BrdU (BD Pharmingen, San Diego, Calif.), in a final concentration of 10 μM. Cells were then harvested by trypsinization, counted and washed with PBS 3 time. Cells were then fixed in 70% ethanol, washed again and denatured with 2M HCl. After neutralization with 0.1M sodium borate solution and 3 washes, cells were incubated with FITC labeled monoclonal antibody to BrdU (Caltag, Burlingame, Calif.), washed again and analyzed by flow cytometry using FACS Calibur (Becton Dickinson).

### Total Cell Count

The total number of viable tumor cells in final cultures was determined by trypan blue dye (Cellgro) exclusion.

### Detection of IGF-BP3 by Western Blot Analysis

Plasma, 5 μl 10 diluted in Tris-Glycine loading buffer was electrophoresed on 18% Tris-Glycine pre-cast gels (Invitrogen, Carlsbad, Calif.) and transferred to a supported nitrocellulose membrane (Bio-Rad> Hercules, Calif.). Membranes were then blocked with 3% milk, incubated with a polyclonal biotinylated antibody to human IGF-BP3 (R&D Systems, Minneapolis, Minn.), washed with PBS, incubated with peroxidase labeled streptavidin (BD Pharmingen) and washed again. Membranes were developed using ECL reagent (Buckinghamshire, England) and an X-ray film.

### Other IGF-BP3 Experiments

Neutralizing antibody to IGF-BP3 (human IGFBP3 specific goat IgG produced in goats immunized with purified, NSO-derived, recombinant IGFBP3) : (R&D Systems, Minneapolis, Minn.) was added to the tumor cell cultures in final concentration 10 μg/ml. Recombinant purified human IGF-BP3 (rhIGF-BP3: Purified, with Phenyl-Sepharose chromatography, Gel Filtration, IGFBP3 affinity chromatography, Reverse Phase HPLC; MW=−47,000 Da) (Upstate Biotechnology, Lake Placid, N.Y.) was added to HT29 cells plated as previously described in final, concentration 100-750 ng/ml in serum free medium. When added to wells containing 10% human serum, rhIGF-BP3 was added in final concentration 750 ng/ml.

### Statistical Analysis

Data are expressed as means±SD. Wilcoxon's matched-pairs signed-ranks test and Spearman's correlation tests were used for statistical analysis.

### Results

### In Vitro Tumor Cell Proliferation

TABLE 3
Mitogenic Activity of Plasma from the OS and LS
Groups

| | | | Plasma Mitogenic Activity | | | |
|---|---|---|---|---|---|---|
| | | | | | Cells recovered from | |
| | | Age | BrdU + cells, % | | culture ×10⁵ | |
| Patient Groups | N | Yrs. | PreOP | POD1 | PreOP | POD1 |
| OS, All Patients | 45 | 56.6 ± 15.8 | 34.2 ± 17.9 | 42.4 ± 19.5** | 5.6 ± 1.6 | 7.0 ± 1.8** |
| OS, Colon Cancer^{a} | 20 | 65.4 ± 12.6 | 30.5 ± 19.1 | 36.3 ± 18.0** | 5.4 ± 1.7 | 6.7 ± 1.8** |
| OS, Obesity | 13 | 43.1 ± 10.9 | 48.5 ± 7.8 | 56.7 ± 6.4** | 6.7 ± 0.6 | 7.8 ± 0.8** |
| OS, Colon Adenoma | 8 | 50.2 ± 15.0 | 23.7 ± 14.3 | 33.8 ± 22.3* | 4.3 ± 1.5 | 6.3 ± 3.0* |
| OS, Diverticulitis | 4 | 69.2 ± 6.2¶ | 26.7 ± 17.4 | 43.9 ± 29.1¶ | 5.7 ± 1.6 | 8.0 ± 1.7¶ |
| LS, All Patients | 39 | 59.8 ± 19.3 | 37.2 ± 18.1 | 36.6 ± 18.9 | 5.2 ± 1.3 | 5.1 ± 1.7 |
| LS, Colon Cancer^{a} | 22 | 63.9 ± 17.6 | 32.6 ± 19.9 | 31.6 ± 18.4 | 4.9 ± 1.4 | 4.8 ± 1.7 |
| LS, Obesity | 8 | 38.9 ± 14.7 | 47.0 ± 14.9 | 42.1 ± 15.9 | 5.9 ± 1.3 | 5.8 ± 1.6 |
| LS, Colon Adenoma | 5 | 74.8 ± 8.3▪ | 49.6 ± 8.7 | 56.5 ± 16.5¶ | 5.4 ± 1.0 | 5.8 ± 1.9¶ |
| LS, Diverticulitis | 4 | 60.8 ± 17.6¶ | 34.9 ± 17.5 | 36.3 ± 23.3¶ | 4.9 ± 0.7 | 5.0 ± 0.8¶ |

*P < 0.05;
**P < 0.005 PreOP versus POD1 using Wilcoxon's matched-pairs signed-ranks test.
▪p < 0.05 compared to identical OS subgroup.
¶Insufficient n for a statistical analysis.
^{a}Patients with colon cancer stage I-III were included; distribution of stages was comparable in OS and LS groups.

### Open Surgery Group POD1 vs PreOp (Table 3)

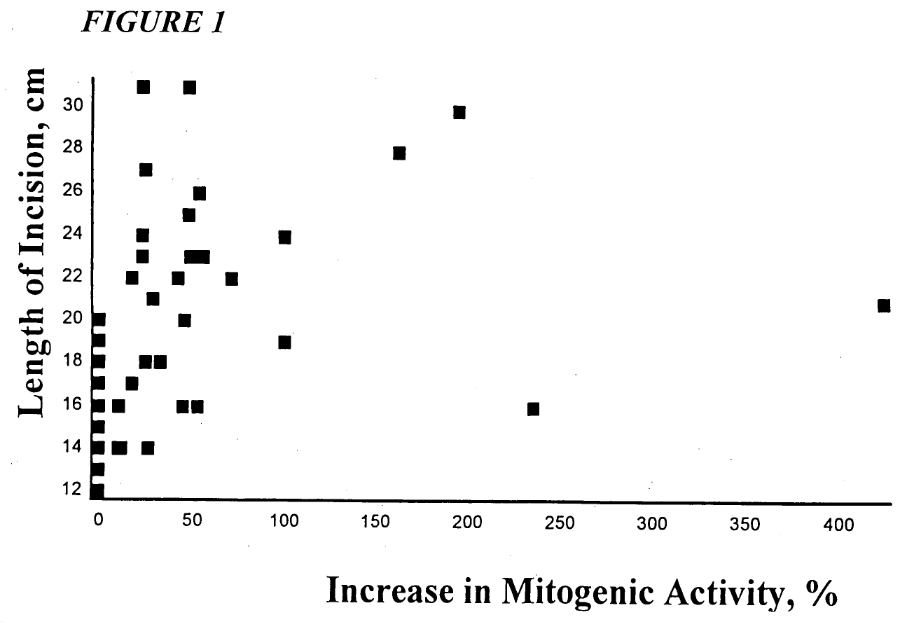

For the overall group, a significantly higher proportion of tumor cells were BrdU+ in the cultures where POD1 OS plasma had been added (42.4±19.5%) when compared to the PreOP OS plasma results (34.2±17.9%, p<0.005). In regards to the total number of viable tumor cells found at the end of the incubation period, significantly more cells (7.0±1.8×10⁵) were noted in the POD1 OS wells than in the PreOP OS wells (5.6±1.6×10⁵, p<0.005) when the OS group as a whole was considered. Similarly, significant differences for both proliferative parameters were noted when the colon cancer and the morbid obesity subgroups were considered separately. The other subgroups were too small to permit statistical analysis. The increase in mitogenic activity of POD1 OS plasma correlated with the length of surgical incision (p<0.01, r=0.58) (FIG. 1). Thirteen OS POD3 plasma samples were assessed. When considered together, no significant differences in BrdU incorporation or total cell count were noted when the POD3 and the PreOp plasma results were compared. However, when 5 patients with an incision equal or greater than 23 cm were considered, significantly increased HT29 proliferation was noted with the POD3 plasma when compared to the PreOP results (data not shown).

### Laparoscopic Surgery Group

No differences in the percentage of BrdU+ cells or the total number of tumor cells were noted when the POD1 LS data were compared to the preOP LS data (Table 3). This was true for the overall group and for the 2 main subgroups where analysis was possible. A total of 14 POD3 LS- plasma samples were similarly assessed. No differences in proliferation were noted when the POD3 LS and PreOp LS data were compared.

### Plasma Factor Characterization Studies

### Initial Studies

In a subset of patients, blood samples were collected in heparinized tubes in addition to the EDTA containing tubes. Although increased HT29 proliferation was noted with POD1 OS vs PreOP OS EDTA samples, no differences in tumor growth were noted when the heparinized plasma was tested (data not shown). In addition, heating of plasma to 99° C. eliminated the effect, which suggested that the factor(s) was a protein. A protein serum factor was searched for which could be stabilized by EDTA. Insulin-like growth factor binding protein 3 (IGF-BP3) was a likely candidate, because EDTA inhibits activity of IGF-BP3 specific serum protease, an enzyme that cleaves IGF-BP3 (Bang, P., et al.). In addition, surgery has been reported to induce IGF-BP3 protease activity (Davenport, M. L., et al.). Because the antibody in the available ELISA for IGF-BP3 reacts both with the intact protein (˜40 kDa) and the biologically inert protein fragments, a Western Blot analysis was performed.

### Western Blot Analysis

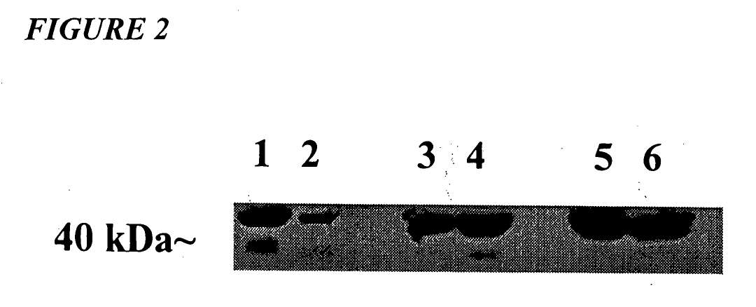

PreOP and POD1 plasma levels of IGF-BP3 were determined for all patients. In 5 of 45 OS patients (11.1%) and 6 of 39 LS patients (15.4%), IGF-BP3 was not detected in any of the samples. For the remaining patients, a decrease in plasma IGF-BP3 on POD1 when compared to PreOP levels was noted in 80.9% of OS patients and in 16.7% of LS patients. OS patients with preserved post-operative levels of circulating IGF-BP3 had shorter incisions (<23cm). Representative Western Blot results are displayed in FIG. 2.

### IGF-BP3 Effect on HT 29 Growth

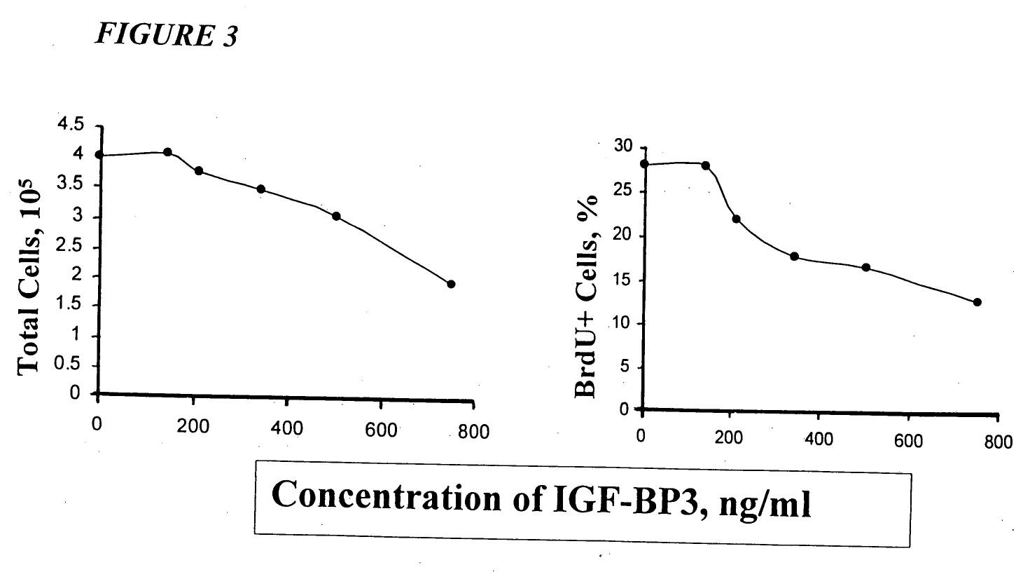

To test whether IGF-BP3 directly affects HT29 growth, human recombinant IGF-BP3 (rhIGF-BP3) was added to serum free cultures of HT29 cells (FIG. 3). RhIGF-BP3 had an inhibitory effect on HT29 cell proliferation in the concentration range of 200-750 ng/ml; higher concentrations have not been tested. IGF-BP3 concentrations lower than 200 ng/ml did not have an impact on cell proliferation.

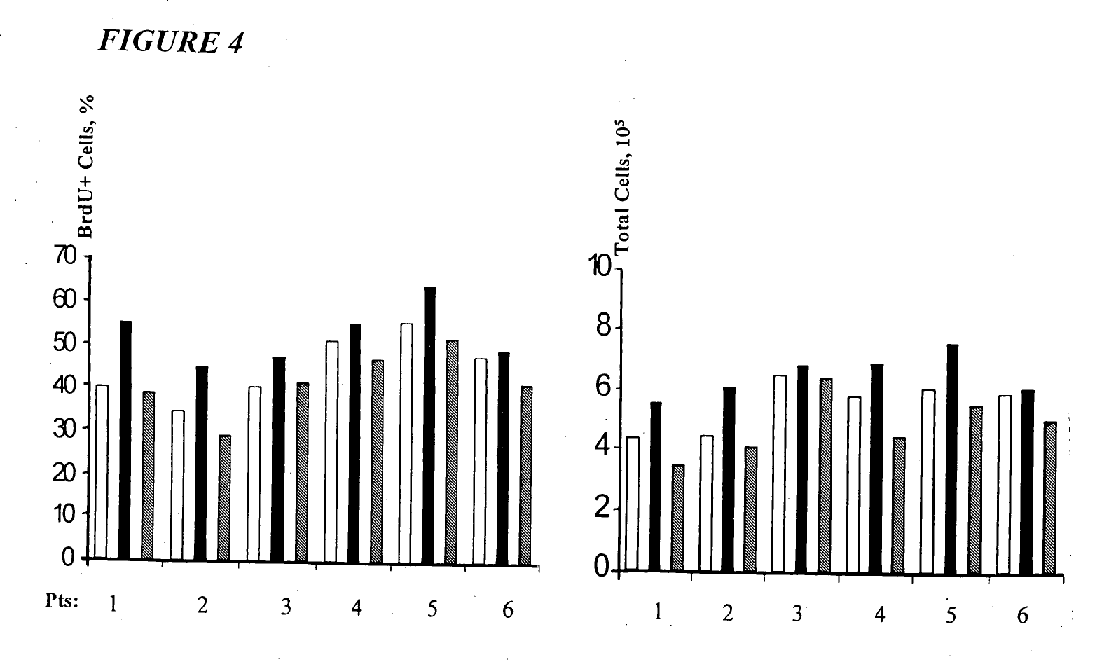

RhIGF-BP3 was added to POD1 OS plasma samples (n=6) at concentration of 750 ng/ml and HT29 proliferation assessed. Significantly less proliferation, as judged by both BrdU assay and cell counts, was noted in the rhIGF-BP3 augmented wells (FIG. 4) when compared to POD1 OS plasma results. There was no significant difference between the supplemented POD1 and the PreOP OS plasma results.

### Impact of Neutralizing Antibody to IGF-BP3

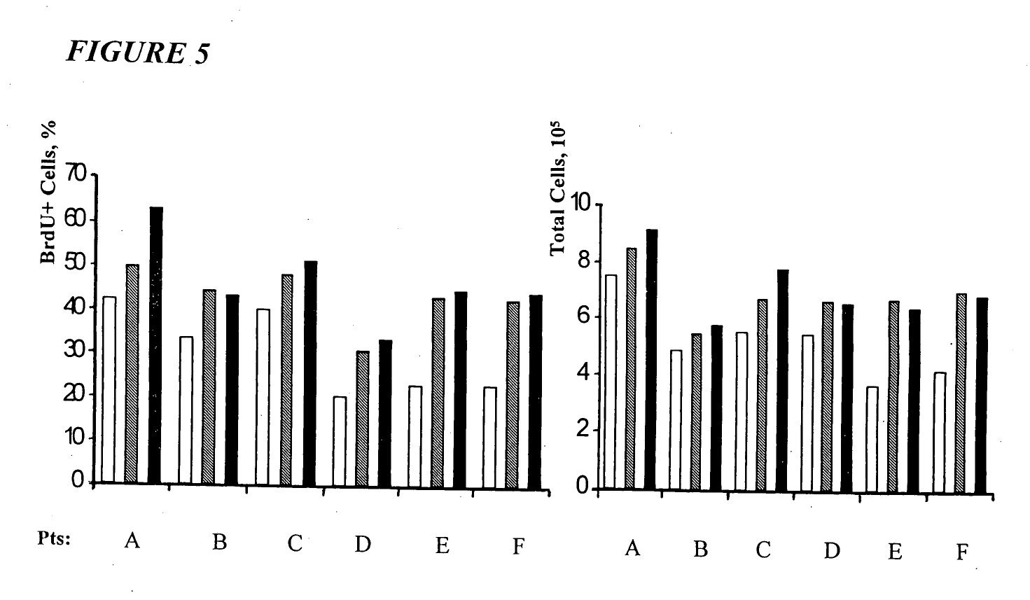

Anti-IGF-BP3 antibody was added to PreOP OS plasma and HT29 proliferation assessed (n=6). Both BrdU and cell count results demonstrated significantly increased proliferation for the antibody supplemented group when compared to the PreOP plasma results (p<0.05). The anti-IGF-BP3 results were similar to those noted with the POD1 OS plasma (FIG. 5).

### Discussion

The present human study was undertaken to determine if major open and closed abdominal surgery had a similar effect on human plasma mitogenic activity for colon cancer cells. In a murine model, open surgery induced an increase in serum mitogenic activity and platelet-derived growth factor was thought to be the responsible protein (Lee, S. W. et al., 2001). The purpose of this human study was to determine if major abdominal surgery carried out via open or laparoscopic means was associated with alterations in the composition of plasma such that in vitro tumor growth would be enhanced. If such an effect was indeed observed, the responsible factor(s) were hoped to be identified.

The results suggest that post-op day 1 plasma from open surgery patients enhances in vitro tumor growth and that the increased proliferation correlated directly with the length of the incision. Post-operative plasma from laparoscopic surgery patients did not have this effect. Increased growth was noted after both open colorectomy and gastric bypass and, therefore, does not appear to be related to the organ being operated on or to the presence of a malignancy. Although not randomized, close scrutiny of the open and closed groups in regards to demographics, associated illnesses, body habitus, indication, and pathology results (for tumors) suggested the two groups were similar. Of note, proliferation studies of the POD3 plasma, carried out on a fraction of the study patients, suggested that the tumor stimulatory effect is lost in all but those open patients with incisions equal or greater than 23 cm.

Plasma from laparotomized mice has been shown to stimulate in vitro tumor growth when compared to results with preoperative plasma. This study assessed the effect of plasma from patients that underwent major open (OS) or laparoscopic surgery (LS) on in vitro tumor cell growth. Eighty-four patients undergoing major abdominal surgery were studied (45 OS, 39 LS). Peripheral blood was collected preoperatively (PreOP) and on days 1(POD1) and 3(POD3) after surgery. HT29 human colon cancer cells were plated with samples of the plasma.

Proliferation was assessed via cell counts and the BrdU incorporation assay. IGF-BP3 (insulin-like growth factor binding protein 3) was detected in plasma via Western Blot analysis. Increased mitogenic activity was noted in POD1 OS plasma when compared to PreOP OS plasma results (p<0.005). This increase correlated with the length of incision (r=0.58, 30 P<0.01) . No differences were noted when the PreOP LS and POD1 LS results were compared or for any of the POD3 vs PreOP comparisons. Hence, major open surgery is associated with alterations in plasma composition that promote HT29 tumor cell proliferation in vitro. As shown, this effect was due, at least in part, to surgery-related depletion of IGF-BP3 in peripheral blood.

Plasma from patients undergoing major open surgery stimulates in vitro tumor growth. Lower IGF-BP3 levels may, in part, account for this change. Plasma from mice undergoing laparotomy has been shown to stimulate in vitro tumor growth. The goals of this study were to determine the effect of plasma from patients that underwent major open (OS) or laparoscopic surgery (LS) on in vitro tumor growth and, if surgery-related differences were noted, to identify the responsible factor(s).

Materials: A total of 58 patients undergoing major abdominal surgery were studied (34 OS and 24 LS patients). Peripheral blood was collected in heparinized and EDTA tubes before surgery (PreOP) and on days 1(POD1) and 3(POD3) after surgery. Plasma was obtained by centrifugation and stored at −70° C.

Proliferation Assay: HT29 human colon cancer cells were plated with 10% human serum from the patients. The BrdU cell proliferation assay was used. IGF-BP3 (insulin-like growth factor binding protein 3) was detected in plasma by Western Blot Analysis using specific antibody. Statistical analysis was performed using paired Student's test and Pearson correlation coefficient. [P value of 0.05 or less was considered statistically significant.]

Increased mitogenic activity was noted with the POD1 OS plasma (41.4±20.4% BrdU+ cells) when compared to results with the PreOP OS plasma (32.5%±17.9%, p<0.01). This increase correlated with the length of incision (r=0.328, P=0.036). No difference in mitogenic activity was noted when the LS PreOP and the LS POD1 results were compared (32.6±20.7% vs 31.7±20.8%, respectively). [No differences were noted when POD3 and PreOp results were compared for either the OS and LS groups.] OS associated stimulation of HT29 cell growth was stronger with EDTA than with heparinized plasma. A serum factor was searched for that might be stabilized by EDTA. EDTA blocks IGF-BP3-related plasma proteolytic activity. Via Western Blot analysis, less IGF-BP3 was noted in the POD1 plasma samples associated with higher mitogenic activity. Purified IGF-BPS at a concentration 500 ng/ml and higher appeared to inhibit HT29 proliferation, while addition of IGF-BP3 neutralizing antibody to PreOP plasma increased its mitogenic activity to the level of POD1 plasma. Major open surgery appears to enhance the ability of human plasma to promote HT29 tumor cell proliferation in vitro. This effect may be due, in part, to depletion of IGF-BP3 in peripheral blood following open surgery.

Having demonstrated the effect, the responsible factor(s) were then identified. Serum PDGF β levels were determined via ELISA, however, no differences were noted (data not provided) in either group. For the reasons stated, IGF-BP3 was a reasonable candidate. The Western Blot findings and the results of the studies that supplemented with rhIGF-BP3 or anti-IGF-BP3 strongly suggest that IGF-BP3 is the responsible factor. An open surgery-related decrease in IGF-BP3 levels, most likely, accounts, in large part, for the in vitro tumor growth differences noted with POD1 Open surgery plasma. It has previously been shown that major open abdominal surgery induces proteolysis of circulating IGF-BP3 (Cotterill, A. M. , et al.)

Intact IGF-BP3 can influence tumor growth via 2 mechanisms. First, it can bind circulating IGF-I, a well known growth factor, and thus limit IGF-I related stimulatory effects (Yu, H., et al.). Secondly, IGF-BP3 itself can deter proliferation directly by inducing tumor cell apoptosis. This direct effect has been documented for prostate cancer (Rajah, R., et al.), breast cancer (Fanayan, S. et al.) and hepatocellular carcinoma cells (Murakami, K., et al.). Interestingly, IGF-BP3's effect on colon cancer cells is less clear; one study suggested it was inhibitory (MacDonald, R. G., et al.) while another reported stimulatory effects (Kansra, S., et al.). In this study, the HT29 colon cell line was shown to be inhibited by IGF-BP3.

The possible implications of these results are far reaching. For the first time, in humans, the choice of surgical approach has been associated with host alterations that may increase the chances that tumor cells in the blood will survive and form a metastases. Thus, IGF-BP3 replacement in open cancer patients or supplementation in closed surgery patients should lower the risk of tumor recurrence.

Insulin-like growth factor binding protein 3 (IGF-BP3) is a serum protein that can exert an inhibitory effect on the growth of tumor cells via 2 major mechanisms: 1) it binds a major cell growth factor, insulin-like growth factor 1 (IGF-1) and 2) it directly inhibits tumor cell growth. The inhibitory effect of IGF-BP3 has been demonstrated for prostate, breast, and lung cancer. It was shown that high doses of IGF-BP3 also directly inhibit colon cancer cell growth in vitro.

In the described study, patients that underwent major open surgery (cancer and non-cancer patients) were noted to have reduced levels of IGF-BP3 shortly after surgery. It was found that plasma from open surgery patients collected on postoperative day 1 stimulated in vitro tumor growth of colon cancer cells when compared to results obtained with their preoperative plasma samples. Addition of IGF-BP3 neutralizing antibody to preoperative plasma resulted in accelerated tumor cell growth to the same degree as observed with postoperative plasma. In addition, supplementing the postoperative day 1 plasma with recombinant IGF-BP3 eliminated the tumor cell stimulation that had been noted with the “raw” postoperative day 1 plasma. Thus, inhibition of recurrent tumor and/or metastatic tumor formation via perioperative administration of insulin-like growth factor binding protein 3 in cancer patients (all types of cancer) undergoing major surgery via a traditional open (a single lengthy incision) surgical approach was proposed.

The surgical resection of cancers is associated with the release of tumor cells into the circulation in a significant proportion of patients. These blood borne tumor cells may give rise to distant metastases. The chances that a circulating tumor cell will successfully form a metastases, regardless of the cancer type, in a patient undergoing an open surgery will be smaller if immediately after surgery the patient receives one of several injections of IGF-BP3.

Prior to this study, it was not known that open surgery induces the partial depletion of circulating plasma IGF-BP3 and that the depleted postoperative plasma stimulates cancer cell growth. Also, this study demonstrates that a similar effect can be achieved by adding IGF-BP3 neutralizing antibody to the preoperative plasma. This study also shows that higher concentrations of recombinant IGF-BP3 improve the inhibitory effect for, e.g., colon cancer cells.

### Experimental Details Part 2

It was previously shown that an important cell growth regulatory protein, insulin like growth factor binding protein 3 (IGFBP-3) is depleted in peripheral blood following open but not laparoscopic surgery. It was also demonstrated that IGFBP-3 induces apoptosis of human colon cancer cells in vitro. Here, the effect of IGFBP-3 on growth of colonic epithelial cells in vivo is reported.

Methods Two tumor models were used: chemically induced carcinogenesis with azoxymethane (AOM) and inoculation of syngeneic colon cancer cells. In AOM induced carcinogenesis, wild type (WT) and IGFBP-3 transgenic (IGFBP3-TG) CD1 mice were injected with AOM and the number of aberrant crypt foci (ACF) in the colon studied. In syngeneic model BALB/c mice were inoculated with CT26 cells. Control group received saline while test group was administered with IGFBP-3 weekly. Tumor weight was assessed 2.5 weeks after establishment.

Results The number of ACF was significantly lower in IGFBP-3 transgenic mice, 1.3±1.1 compared to WT controls, 6.8±6.0 (p<0.001). Further, CT26 tumors were significantly smaller in BALB/c mice that received IGFBP-3, 0.364±0.165g than in control mice, 0.742±0.261 g (p<0.01).

Conclusions IGFBP-3 inhibits the development of colonic tumors in experimental models and may hold promise as an adjuvant therapy for patients with neoplasms.

### Introduction

Colon cancer is one of the leading causes of tumor related death. Surgical removal of the primary tumor combined with adjuvant chemotherapy for subset of patients is the standard treatment for colon cancer patients. Unfortunately, despite these measures there is a reasonably high recurrence rate and the overall 5 year survival is about 55 percent. It has been noted in experimental studies that open surgical (OS) trauma is associated with an increased rate of tumor growth and establishment in the early postoperative period. One potential mechanism for surgery-related alterations in tumor growth is depletion of cell growth regulatory serum proteins via surgical trauma induced proteolysis. In a previously published study, it was demonstrated that in cultures supplemented with postoperative plasma from OS patients, tumor cells had greater proliferative capacity than in cultures with preoperative plasma (Kirman, et al. 2002).

IGFBP-3, originally discovered as an insulin growth factor 1 (IGF-I) binding protein, was thought to exert its tumor suppressive properties by binding and limiting availability of IGF-I, a crucial growth stimulator, to the cells (Cubbage, et al.). It was later determined that IGFBP-3 has its own independent tumor growth suppressive properties; the mutated form of protein which is unable to bind IGF-I or IGF-II, still inhibits tumor growth (Hong, et al.). In addition, IGFBP-3 induces tumor cell apoptosis even in the absence of IGF-I receptors (Valentinis, et al.). The precise cellular source of IGFBP-3 is not known, cells of different origin have been reported to produce this molecule (Reeve, et al.). In peripheral blood, IGFBP-3 circulates in 2 major forms, the intact bioactive (in regards to its cell growth regulatory activity) 43-45 kDa protein and its inactive 30 kDa degradation products (Grimberg, et al.). A great variety of proteases have been found to cleave the IGFBP-3 molecule. Activation of these proteases may depend on inflammatory processes. Thus, the pro-inflammatory cytokine IL-6 has been reported to induce IGFBP-3 proteolysis (De Benedetti, et al.) and a correlation between the postoperative IGFBP-3 depletion and 1L-6 concentration in OS patients was found (Kirman, et al. 2004).

IGFBP-3 affects cell growth through the induction of apoptosis and inhibition of DNA synthesis (Grimberg, et al.). The in vitro growth suppressive action of IGFBP-3 has been reported for human breast, prostate, colon cancer as well as for other types of tumor cells (Kirman, et al. 2002; Gill, et al.; Boyle, et al.). Human IGFBP-3 also suppresses the growth of murine cells (Cohen, et al.). In clinical studies an association between a decreased concentration of circulating IGFBP-3 and the risk for the development of colon cancer has been found (Ma, et al.; Palmqvist, et al.). The aim of the present work was to study the effect of IGFBP-3 on the in vivo development and growth of neoplastic colonic epithelial cells using 2 experimental models.

### Materials and Methods

### Mice

Six week old female BALB/c mice were purchased from Jackson Laboratories (Bar Harbor, Me.). CD1 mice carrying human IGFBP-3 cDNA (IGFBP3-TG) (Modric, et al.) were the kind gift of Dr. L. J. Murphy (Department of Medicine, Winnipeg, Canada). The transgenic colony was expanded and the presence of circulating serum human IGFBP-3 (hIGFBP-3) screened in ELISA. Male IGFBP3-TG mice were 8 weeks old at the beginning of the experiment. The control wild type male 8 week old CD1 (WT) mice were purchased from Charles River Laboratories (Wilmington, Mass.).

### Cells

CT26 murine colon adenocarcinoma cells (American Type Culture Collection, Manassas, Va.) were maintained in RPMI-1640 medium (Mediatech Inc. Herndon, Va.) supplemented with 10% fetal bovine serum (Sigma, St. Louis, Mo.), 2 mM L-glutamine (Mediatech Iric), 10 mM HEPES (Mediatech lnc) and 1 mM sodium pyruvate (Mediatech lnc).

### In vitro Effect of Recombinant Human IGFBP-3 on Growth of CT26 Cells

CT 26 cells were plated 8×10⁵/well in 6-well Costar tissue culture plates (Corning Inc., Corning, N.Y.) in complete RPMI-1640 medium and allowed to adhere. Subsequently, cells were washed twice with serum free RPMI medium. Non-glycosylated E. coli expressed recombinant hIGFBP-3 bioactive fragment (rhIGFBP-3, Upstate USA Inc., Charlottesville, Va.), 10 μg/ml in serum free RPMI 1640 was then added to the test wells. Cells were incubated for 48 hours and subsequently harvested by trypsinization, counted, stained with AnnexinV-FITC (BD Biosciences, San Jose, Calif.), propidium iodide (PI) and analyzed by flow cytometry. Apoptosis is associated with externalization of phosphatidylserine (PS), while necrosis is associated with an increased cell membrane permeability. Therefore, apoptotic cells can be identified with a labeled PS binding protein, Annexin V, while necrotic cells will accumulate a DNA binding dye, PI. The number of live cells in samples was determined by multiplying the number of total cells by the proportion of AnnexinV-Pl-cells.

### The in vivo Administration of rhIGFBP-3

CT26 cells were harvested, washed 3 times in PBS and inoculated subcutaneously in BALB/c mice, 10⁵/mouse. Concomitantly with the cell injection, the test group (n=11) received rhIGFBP-3 (Upstate USA), 50 μg/mouse while the control group (n=10) received solvent alone. The test group received two more peritumoral injections of rhIGFBP-3 at 7 day intervals, 100 μg/mouse whereas the control group were given comparable injections of solvent alone according to the same schedule. Seventeen days after the tumor cells were injected, the mice were sacrificed, and their tumors excised and weighed. The presence of CT26 adenocarcinoma cells in excised tissue mass was confirmed by assessment of cytokeratin expression. Briefly, a single cell suspension was prepared from the excised mass. Cells were then ethanol fixed, washed in PBS, stained with FITC labeled antibody to mouse pan-cytokeratin (Sigma) and analyzed by flow cytometry.

### The Effect of IGFBP-3 Over-Expression on Development of Chemically Induced Tumors

IGFBP-3 TG (n=14) and WT mice (n=14) were injected with azoxymethane (AOM)(Sigma) 10 mg/kg intraperitoneally. Six weekly injections of AOM were given; the mice were sacrificed 6 months after the last injection. Subsequently, the whole colon was removed, opened longitudinally, flushed with PBS and fixed in buffered 10% formalin between 2 sheets of filter paper. Forty eight hours later, tissue was stained with 0.2% methylene blue (LabChem Inc., Pittsburgh, Pa.) solution in formalin and visualized using an inverted microscope with a ×10 objective. Aberrant crypt foci (ACF) were recognized by their increased size, intensive staining and an elevated appearance.

### IGFBP-3 Assays

The concentration of total IGFBP-3 was assessed using an ELISA kit (Diagnostic Systems Laboratories Inc., Webster, Tex.) according to the manufacturer's instructions and a microplate reader, ELx800 (Bio-Tec, Virginia Beach, Va.). The integrity of IGFBP-3 molecule in cultures was tested using the Western Blot analysis of immunomagnetically selected products. Briefly, aliquots of CT26 cell supernatants from: 1) control CT26 cells, 2) CT26 cells cultures supplemented with rhIGFBP-3 and 3) rhIGFBP-3 in a cell free medium, were incubated with a polyclonal biotinylated antibody to IGFBP-3 (R&D Systems, Minneapolis, Minn.) and with streptavidin coated Dynabeads (Dynal, Oslo, Norway). Immunomagentically isolated products were placed in Tris-Glycine SDS sample buffer (lnvitrogen, Carlsbad, Calif.) containing β-mercaptoethanol, heated, separated under denaturing conditions using 18% pre-cast gels (Invitrogen) and transferred to a nitrocellulose membrane (Bio-Rad Laboratories, Hercules, Calif.). The membranes were blocked with 6% milk, incubated with a mouse antibody to human IGFBP-3 (R&D Systems), after which HRP labeled antibodies to mouse immunoglobulins (Pierce, Rockford, Ill.) were added. Finally, the reaction was developed using a chemiluminescent detection kit (Pierce) and the membranes were exposed to an X-ray film.

### Statistical Analysis

The results are presented as mean±SD values. Statistical analysis has been performed using the Mann-Whitney test.

### Results

### The Effect of rhIGFBP-3 on the Growth of CT26 Cells in vitro

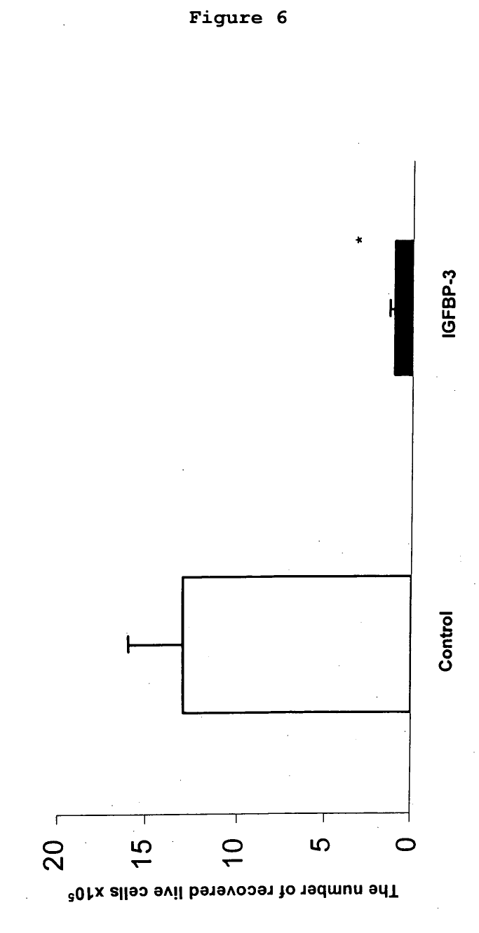

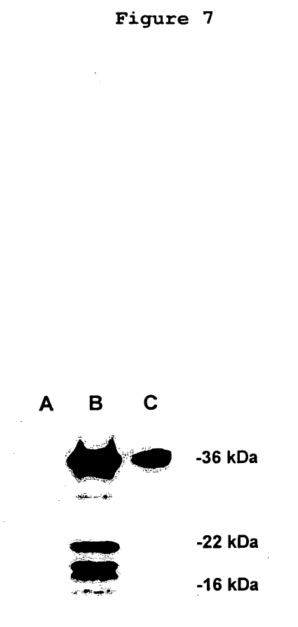

Exposure of CT26 cells to IGFBP-3 significantly suppressed growth of CT26 cells. The mean number of recovered live cells from wells with rhIGFBP-3 was significantly lower than from control wells (p<0.01, FIG. 6). Cell processing of rhIGFBP-3 was also assessed. CT26 cells induced noticeable proteolysis of rhIGFBP-3 in 2-day cultures (FIG. 7).

### The Effect of rhIGFBP-3 on Establishment and Growth of CT26 Tumors

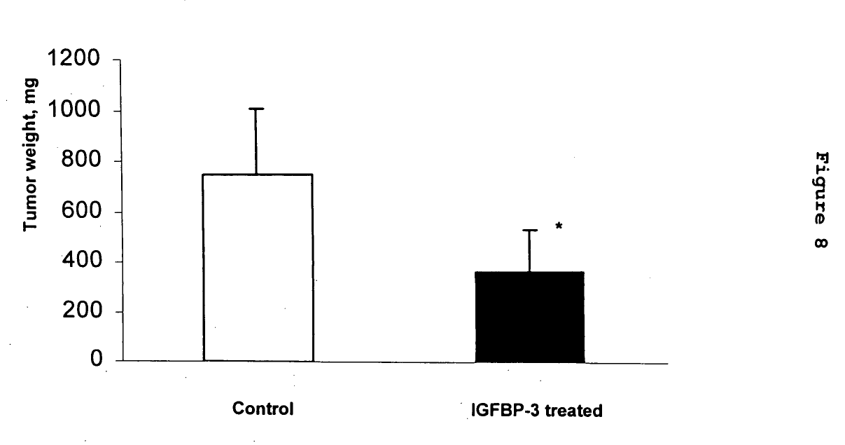

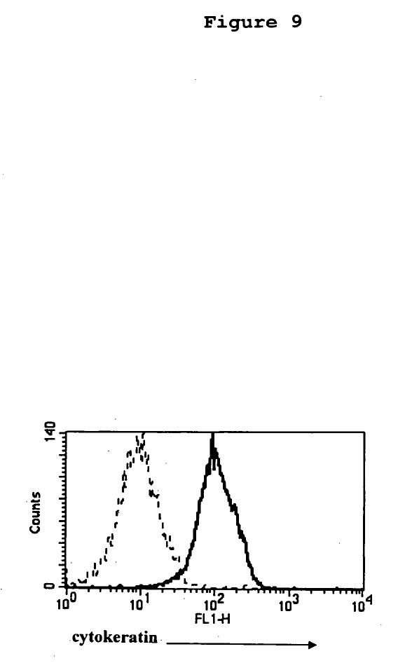

Injections of rhIGFBP-3 were administered to the test mice (n=11) and solvent alone to the control mice (n=10) 1) concomitantly with CT26 tumor cells, 2) on day 7 and 3) on day 14 after the first injection. On day 17, the mean tumor weight was significantly lower in IGFBP-3 treated mice, 364±165 mg than in controls, 742±261 mg (p<0.01, FIG. 8). Cells in the subcutaneous tumor masses excised from the mice were of epithelial origin as they all expressed cytokeratin (FIG. 9).

### The Effect of JGFBP-3 Over-Expression on Development of Chemically Induced Tumors

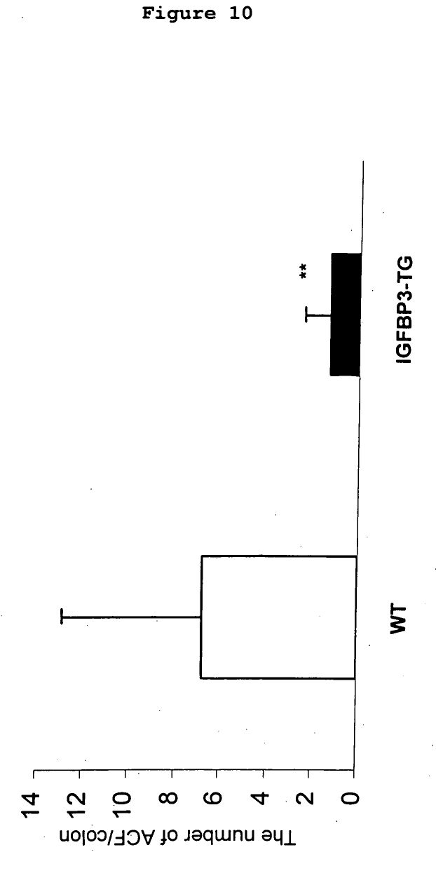

WT (n=14) and IGFBP3-TG mice (n=14) were used in this azoxymethane (AOM) induced carcinogenesis study. Human IGFBP-3 was undetectable in plasma from the WT mice whereas the mean hIGFBP-3 concentration in the plasma from IGFBP3-TG mice was 4800±1428 ng/ml. The mean weight of colonic tissue was significantly lower in the IGFBP3-TG mice, 368.8±60.45 mg than in WT mice, 633.6±54.18 mg (p<0.001). Similarly, total body weight was decreased in IGFBP3-TG mice, 31.6±1.3 g compared to the WT mice, 45.5±1.2 g (p<0.001). However, when corrected for the difference in total body weight, the colonic tissue mass was slightly, albeit significantly, reduced in IGFBP3-TG mice (p<0.05) . The number of AOM induced aberrant crypt foci (ACF) per colon was fewer in IGFBP3-TG, 1.3±1.1 mice than in WT mice, 6.8±6.0 (p<0.001, FIG. 10).

### Discussion

The tumor growth inhibitory effect of IGFBP-3 has been demonstrated for various types of cells in vitro; the current study investigates the in vivo effect of this protein on the development of experimental tumors. It has been shown that IGFBP-3 suppresses growth of not only human, but also of murine cells (Cohen, et al.). The present study deomonstrated that rhIGFBP-3 inhibited the growth of CT26 murine colon adenocarcinoma cells in vitro. Thus, a significantly lower number of viable CT26 cells was recovered from the wells that contained rhIGFBP-3 compared to the control wells. The effect of rhIGFBP-3 on the growth of CT26 tumors in vivo was further studied. Mice that received peritumoral injections of rhIGFBP-3 developed significantly smaller tumors than control mice. This suggests that IGFBP-3 inhibits the growth of CT26 tumors in vivo. The inhibitory effect, although statistically significant, was not dramatic. Of note, the in vitro study showed that CT26 cells induce proteolysis of IGFBP-3. This effect, most likely, is not IGFBP-3 specific, nor is it a CT26 cell specific effect. Many other types of cells, both transformed and non-transformed, are able to degrade IGFBP-3 (Salahifar, et al.; Manes, et al.; Irwin, et al.) via enzymatic proteolysis. Enzymatic cleavage of IGFBP-3 often involves the molecule's bioactive domains (Claussen, et al.); as a result, biologically inactive degradation fragments are generated. It is therefore possible that the in vivo effect of rhIGFBP-3 is diminished secondary to its degradation by CT26 cells. This may limit the effectiveness of rhIGFBP-3 administration in well established tumors, which contain several millions of CT26 cells. A hIGFBP-3 cDNA transfer system, which will allow the maintenance of high levels of IGFBP-3 due to its local over-expression, has been established.

The effect of IGFBP-3 over-expression on development of colonic tumors using a transgenic murine model was further investigated.

Transgenic mice carrying human IGFBP-3 cDNA under the control of phosphoglycerate kinase (PGK) promoter have been reported to produce high amounts of IGFBP-3 protein; the concentration of circulating IGFBP-3 is similar to human levels Modric, et al). These mice have been also reported to have a reduced body weight (Modric, et al.) which suggests that tissue growth inhibition can be achieved by IGFBP-3 over-expression. In the present study, a decreased body weight in the IGFBP3-TG mice was noted when compared to the WT animals; these results are consistent with previously reported findings. In addition, a decreased colonic weight was observed in the IGFBP3-TG mice. Thus, the IGFBP-3 protein might function as an important growth regulatory factor for colonic cells.

Induction of chemical carcinogenesis by AOM has shown that IGFBP3-TG mice develop a significantly fewer number of ACF than wild type animals. However, in general the number of ACF in CD1 mice was not high. Susceptibility to chemical carcinogens, in particular AOM, show a certain range of variability in different strains of mice. Thus, A/J mice have been reported to be highly susceptible, SWR/J intermediately susceptible and AKR/J resistant to AOM induced carcinogenesis (Papanikolaou, et al. 2000; Papanikolaou, et al. 1998). The CD1 strain used in this study is moderately susceptible to AOM. Several new experimental models which will better permit the assessment of the growth regulatory effect of IGFBP-3 on murine and human tumor cell lines are currently being developed.

In conclusion, IGFBP-3 inhibits the development and growth of colonic tumors in experimental models. This protein holds promise as a possible therapeutic agent for patients with colon cancer. It is possible that administration of IGFBP-3 or blockade of IGFBP-3 proteolysis may reduce disease recurrence in colon cancer patients undergoing colectomy, a procedure that is associated with depletion of endogenous IGFBP-3.

### Experimental Details Part 3

It was earlier shown, via Western Blot analysis, that open (OS) but not laparoscopic surgery (LS) induces a qualitative decrease in plasma IGFBP-3 (insulin-like growth factor binding protein 3) levels on postoperative day 1 (POD1) and that POD1 open plasma stimulates in vitro tumor growth. Intact IGFBP-3 has tumor suppressive effects but its degradation products do not; ELISA inevitably measures both. In this study, using a novel combined Western Blot and ELISA analysis method, precise plasma levels of intact IGFBP-3 on POD2 after open and closed colorectal cancer resection (Stage I-III) were determined.

Methods 15 OS patients (pts), mean incision length 26.7±15.5 cm and 16 LS pts, mean incision length 5.3±3.1 cm were studied. Intact IGFBP-3 levels were calculated via ELISA and Western Blot analysis in plasma collected preoperatively and on POD2.

Results In OS patients the mean preop concentration of intact 43-45 kDa IGFBP-3 protein was 1920±1430 ng/ml; it decreased dramatically on POD2 to 355±545 ng/ml (p<0.005). In the LS group, there was no significant difference noted, 1305±807 ng/ml preop vs 922±714 ng/ml on POD2.

Conclusions Open cancer resection, unlike its minimally invasive alternative, induces a dramatic decrease in concentration of intact IGFBP-3 which may have important implications in regards to colon cancer recurrence.

### Introduction

Colorecatal adenocarcinoma is one of the leading causes of cancer-related death in the world. Surgical removal of the primary tumor remains the treatment of choice. Unfortunately, despite surgical excision, a substantial proportion of patients will develop tumor recurrences. Whereas some recurrences are due to micrometastases already present at distant sites at the time of surgery, others likely develop from blood borne tumor cells that are disseminated into the bloodstream during the operation. Paradoxically, the operation, the goal of which is to remove the primary tumor and cure the patient, may actually temporarily increase the chances that residual tumor cells will survive and form metastases because of surgical trauma related immunosuppression and deleterious alterations in the balance of tumor growth and inhibitory factors.

It was previously demonstrated that following open surgery (OS) but not laparoscppic assisted surgery (LS), the direct mitogenic activity of plasma for human colon cancer cell lines increases and that a decline in the concentration of insulin like growth factor binding protein 3 (IGFBP-3) accounts for, at least, part of this effect (11). IGFBP-3 was originally described as a binding protein of a major cell growth factor, insulin-like growth factor I (IGF-I) (1,5,17), a major cell growth factor. IGFBP-3 exerts its cell growth suppressive effects via 2 major mechanisms, by limiting availability of IGF-I and by inducing a direct pro-apoptotic and DNA synthesis inhibitory effects in a great variety of tumor cells (1,5,10,17,23). IGFBP-3 has been shown to directly inhibit the growth of prostate, breast cancer and colon cancer cells (4,8,11). In addition, IGFBP-3 gene transfer dramatically inhibits the growth of established tumors, such as non-small cell lung cancer, in experimental models (13). Thus, there is a growing body of evidence that IGFBP-3 is an important tumor cell growth inhibitory factor.

Humans have relatively high plasma concentrations of IGFBP-3; in healthy subjects it ranges from 1.5 to 11 μg/ml, according to the ELISA measurements. ELISA detects the sum of the intact 43-45 kDa protein and its smaller degradation products. Only the intact IGFBP-3 protein is bioactive in regards to the tumor cell growth regulatory effects. Thus, IGFBP-3 proteolysis results in degradation products that do not inhibit tumor growth. Various proteases have been shown to cleave IGFBP-3; matrix metalloproteinases 1, 3, and 7 as well as plasmin and thrombin all have the ability to enzymatically degrade IGFBP-3 (2,3,7,19). Metalloproteinase concentrations have been shown to increase during periods of active inflammation in patients with arteritis and rheumatoid arthritis as well as in patients with other inflammatory conditions (18,22). The pro-inflammatory cytokines that are released after surgery, IL-6 for example, are thought to indirectly contribute to the enhanced proteolytic activity observed postoperatively. Specifically, IL-6 overproduction has consistently been shown activate IGFBP-3 proteolytic cleavage (6).

Despite the fact that chronic inflammatory disorders are accompanied by IGFBP-3 degradation, clinical trials that investigated IGFBP-3 levels in patients with active disease and patients in remission as well as in healthy subjects have not found dramatic differences (9). Open surgerical trauma is associated with inflammation. It was previously shown that colon cancer patients undergoing open colectomy experience a significant but not a severe decrease in the level of total circulating IGFBP-3 as measured by ELISA (12). These less than impressive changes in IGFBP-3 levels may be related to the fact that the ELISA detects both the intact IGFBP-3 as well as its degradation products. The aim of the current work was to study the effect of open versus laparosocspic surgery on the concentration of intact IGFBP-3 in the early postoperative period. A combined ELISA and Western blot analysis method has been utilized to achieve this goal.

### Materials and Methods

### Patients

Thirty one patients with colorectal cancer scheduled for OS or LS were included in the study. The OS group contained 15 patients (9 males and 6 females); 6 patients had stage I, 2 stage II and 7 stage III disease. There were a total of 16 patients in the LS group (6 males and 10 females): 4 had stage I, 5 stage II and 7 stage III disease. The average incision length was 26.7±15.5 cm in the OS group and 5.3±3.1 cm in LS group. The breakdown of left, right bemicolectomies and segmental resections in each group was similar, so was the extent of resection in regards to pathological criteria. The operation time was comparable in both groups. Peripheral blood was collected in EDTA tubes from all patients preoperatively and on postoperative days (POD) 1-3.

### IGFBP-3 ELISA

The concentration of total IGFBP-3 was assessed using an ELISA kit (Diagnostic Systems Laboratories Inc., Webster, Tex.) according to the manufacturer's instructions and a microplate reader, ELx800 (Bio-Tec, Virginia Beach, Va.). EDTA Preoperative and postoperative day 2 (POD2) plasma samples were applied in duplicates.

### IGFBP3 Western Blot Analysis Assisted by Immunomagnetic Separation

Total IGFBP-3 protein was immunomagentically separated from the plasma samples. Briefly, EDTA plasma samples (300 μl) were incubated with magnetic beads (Dynal Biotech, Oslo, Norway) coated with the anti-IGFBP3 antibody (R&D Systems, Minneapolis, Minn.). The products were isolated using a magnet and electrophoretically separated on 18% SDS-polyacrilamide gels under reducing conditions. Proteins were transferred to a nitrocellulose membrane. After being blocked with 6% milk, the membrane was incubated with a polyclonal mouse antibody to human IGFBP-3 (R&D Systems) and after several washes-with peroxidase labeled antibody to mouse IgG (Pierce Biotechnology, Rockford, Ill.). After extensive washes, membranes were incubated with a chemiluminescent reagent (Pierce) and exposed to an X-ray film. The ratio of “intact IGFBP3/total IGFBP3” as well as the ratio of “IGFBP3 fragments/total IGFBP3” was determined electronically using Scion Image software. The concentration of intact IGFBP3 was calculated as follows: Intact IGFBP-3 (ng/ml)=Total IGFBP-3 (ng/ml) [ELISA derived concentration]×(intact IGFBP-3/total IGFBP-3)[ratio derived from Western Blot analysis].

### Statistical Analysis

The results are expressed as Mean±SD values. The difference between pre-versus postoperative values within a group was analyzed using the Wilcoxon's test. The difference between different groups of patients was analyzed using a Mann-Whitney test. A P value of 0.05. or less was considered as statistically significant.

### Results

### Total IGFBP-3 Concentration

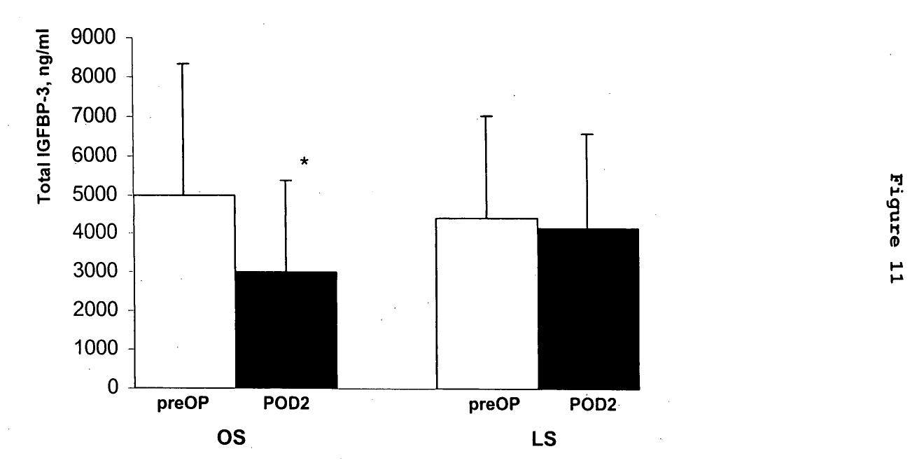

In the OS group, the concentration of total IGFBP-3, as detected in ELISA, was slightly but significantly lower in POD2 EDTA plasma samples, 3008±2348 ng/ml compared to preoperative samples (preOP), 4971±3365 ng/ml (p<0.05) (FIG. 1). In the LS group, the mean preOP IGFBP-3 concentration, 4416±2554 ng/ml was comparable to the mean POD2 IGFBP-3 concentration, 4144±2394 ng/ml (FIG. 11).

### Western Blot Analysis of Plasma IGFBP-3

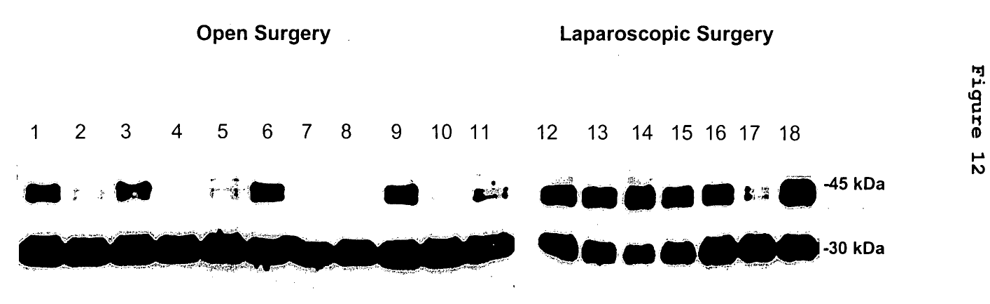

Plasma IGFBP-3 was detected as an intact 43-45 kDa doublet protein and its 30 kDa degradation products (FIG. 12). A dramatic depletion in intact IGFBP-3 was noted in the majority of OS patients; this depletion peaked on POD1, and persisted, to a lesser extent on POD2. By POD3 some restoration of the intact 43-45 kDa protein was observed (FIG. 12). The majority of LS patients did not manifest an obvious qualitative decrease in the intact protein on POD 1-3; however, and of note, in approximately; ⅓ of the LS patients such a decrease was noted on POD 1 and 2.

### Intact IGFBP-3 Concentration

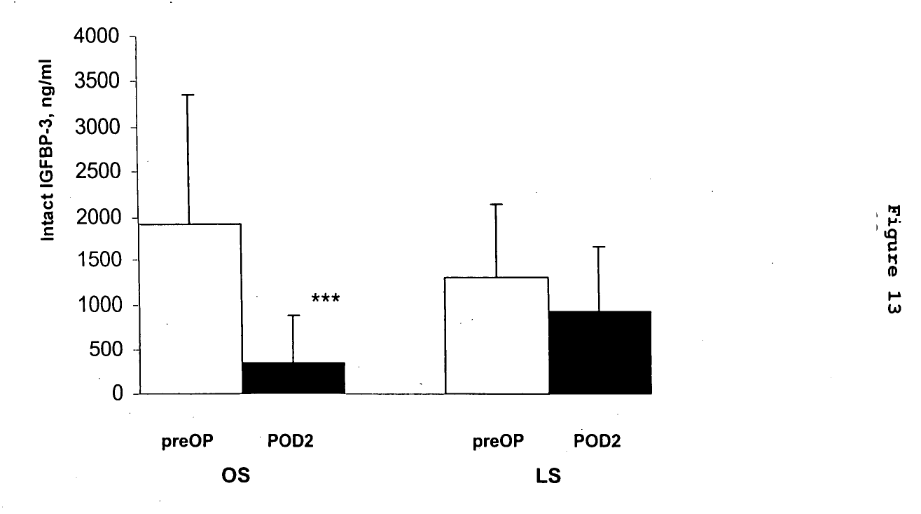

Using the formula mentioned above, the ELISA results and the ratios of intact to total IGFBP-3 levels obtained from the Western blots were used to calculate the levels of intact protein. In OS patients, a dramatic decrease in the level of intact IGFBP-3 was observed on POD2, 355±545 ng/ml compared to preOP values, 1920±1430 ng/ml (p<0.0003) (FIG. 13). The mean intact IGFBP-3 concentration value in the LS group on POD2 (922±714 ng/ml), although less, was not significantly different from the average value calculated for the preOP samples (1305±807 ng/ml, p=0.2). There was a statistically significant decrease in the mean plasma IGFBP-3 levels in OS POD2 samples when compared to the values of LS POD2 samples (p<0.03). The mean plasma IGFBP-3 concentration in preOP samples was comparable between the OS and LS groups (FIG. 13). In regards to the LS group, although the mean IGFBP-3 concentration preoperatively and on POD 2 was not different, 5 out of 16 LS patients (31%) demonstrated a noticeable decrease in the level of intact IGFBP-3 on POD2 when compared to the preOP values. The mean surgery time, incision length and extent of surgery in the subgroup of LS patients that manifested lower POD 2 IGFBP-3 levels were not statistically different compared to the values of these parameters found in the remaining LS patients in whom IGFBP-3 levels were preserved (exact Fisher's test used for this analysis). However, LS patients with low POD2 IGFBP-3 levels tended to have a more advanced stage of the disease; 4 were diagnosed with stage III and 1 with stage II colon cancer versus 4 patients with stage I, 4 patients with stage II and 3 patients with stage III in the rest of the LS group.

### Discussion

It was demonstrated that open but not laparoscopic colectomy induces a dramatic decrease in the concentration of intact IGFBP-3 in colon cancer patients in the early postoperative period. It has been previously shown that recombinant human IGFBP-3 inhibits the growth of human colon cancer cells in vitro (11,12). Therefore, depletion of this important cell growth regulatory factor following open surgery may lead to decreased on the part of the patient to tumor cells in the bloodstream and may result in increased tumor establishment and growth rates. Several clinical trials have been undertaken to evaluate the role of the tumor growth inhibitory protein IGFBP-3 as a risk factor for the development of tumors. Low plasma IGFBP-3 levels and/or an increase in the IGF-1/IGFBP-3 ratio have been shown to be associated with an increased risk of prostate, bladder and colon cancer (14-16,24). No alteration in IGFBP-3 concentration in patients with colorectal adenomas has been noted (21). However, low IGFBP-3 levels may be a predictor factor of colon cancer development in adenoma patients (20). The clinical trials that have investigated the effect of altered plasma IGFBP-3 levels on the development and/or progression of tumors have relied on immunoassays that measure total IGFBP-3 concentration, which includes intact IGFBP-3 and its biologically inactive degradation products. It was shown here that a combined Western Blot and ELISA methos allows calculation of the concentration of the bioactive intact tumor growth inhibitory IGFBP-3 protein. In the current study, the concentration of intact but not total IGFBP-3 was dramatically decreased following open colectomy procedures. It is likely that OS linked inflammatory processes induce activation and/or release of proteases that degrade IGFBP-3. It was previously reported that the level of pro-inflammatory cytokine IL-6 increases following OS and that this increase correlates with IGFBP-3 depletion (12). Further studies are needed to identify the specific proteases that are involved in open surgery associated JGFBP-3 depletion.

The great majority of OS patients manifest a decrease in the concentration of intact IGFBP-3 on postoperative days 1-2. Also, approximately ⅓ of LS patients also show a similar decline in intact IGFBP-3 levels at the same timepoints, although, when the entire cohort of LS patients is assessed, the difference between preoperative and postoperative values is not statistically significant. Presently, it is not known what exactly predisposes LS patients to IGFBP-3 depletion; analysis of this data reveals no correlation between IGFBP-3 levels and duration of operation, incision length, or the type of surgery performed. However, the stage of disease may play a role; a greater proportion of patients with stage III disease had a notable decrease in IGFBP-3 levels after surgery than was observed in patients with stage 1 disease. In conclusion, open colectomy, to a considerably greater extent than its minimally invasive alternative, induces a dramatic decrease in concentration of intact IGFBP-3 in colon cancer patients early after surgery which might have important implications in regards to tumor recurrence and survival. Presently, IGFBP-3 levels have not been correlated with either survival or tumor recurrence.

### REFERENCES

### Experimental Details Part 1 and Background of the Invention

1. Allendorf, J. D.; Bessler, M.; Horvath, K. D.; Marvin, M. R., Laird, D. A.; Whelan, R. L. Increased tumor establishment and growth after open vs laparoscopic surgery in mice may be related to differences in postoperative T-cell function. Surg. Endosc. 1999, 13, 233-235.
2. Allendorf, J. D. ; Bessler, M.; Horvath, K. D.; Marvin, M. R,; Laird, D. A.; Whelan, R. L. Increased tumor establishment and growth after open vs laparoscopic bowel resection in mice. Surg. Endosc. 1998, 12, 1035-1038.
3. Allendorf, J. D.; Bessler, M.; Kayton, M. L.; Oesterling, S. D.; Treat, M. R.; Nowygrod, R.; Whelan, R. L. Increased tumor establishment and growth after laparotomy vs laparoscopy in a murine model. Arch. Surg. 1995, 130, 6, 649-653.
4. Bang, P.; Fielder, P. J. Human pregnancy serum contains at least two distinct proteolytic activities with the ability to degrade insulin-like growth factor binding protein-3. Endocrinology 1997, 138, 3912-3917.
5. Cotterill, A. M.; Mendel, P.; Holly, J. M. P.; Timmins, A. G.; Camacho-Hubner, C; Hughes, S. C. et al. The differential regulation of the circulating levels of the insulin-like growth factors and their binding proteins (IGFBP) 1, 2 and 3 after elective abdominal surgery. Clin. Endocrinol. 1996, 44, 91-101.
6. Da Costa, M. L.; Redmond, P.; Bouchier-Hayes, D. J. The effect of laparotomy and laparoscopy on the establishment of spontaneous tumor metastases. Surgery 1998, 124, 516-525.
7. Davenport, M. L.; Isley, W. L.; Pucilowska, J. B.; Pemberton, L. B.; Lyman, B.; Underwood, L. E.; Clemmons, D. R. Insulin-like growth factor-binding protein-3 proteolysis is induced after elective surgery. J. Clin. Endocrinol. Metab. 1992, 75, 590-595.
8. Fanayan, S.; Firth, S. M.; Butt, A. J.; Baxter, R. C. Growth inhibition by insulin-like growth factor-binding protein-3 in T47D breast cancer cells requires transforming growth factor-beta (TGF-beta) and the type II TGF-beta receptor. J. Biol. Chem. 2000, 275, 39146-39151.
9. Kansra, S.; Ewton, D. Z.; Wang, J.; Friedman, E. IGFBP-3 mediates TGF bet 1 proliferative response in colon cancer cells. Int. J. Cancer 2000, 81, 373-378.
10. Lee, S. W.; Gleason, N. R. ; Stapleton, G. S. ; Zhai, C.;

Huang, E. H.; Bessler, M.; Whelan, R. L. Increased platelet-derived growth factor (PDGF) release after laparotomy stimulates systemic tumor growth in mice. Surg. Endosc. 2001, 15, 981-985.
11. Lee, S. W.; Gleason, N. R.; Southall, J. C.; Allendorf, J. D.; Blanco, I.; Huang, E. H. et al. A serum-soluble factor(s) stimulates tumor growth following laparotomy in a murine model. Surg. Endosc. 2000, 14, 490-494.
12. Lee, S. W.; Southall, J. C.; Allendorf, J. D.; Bessler, M.; Whelan, R. L. Tumor proliferative index is higher in mice undergoing laparotomy vs. CO2 pneumoperitoneum. Dis. Colon Rectum 1999, 42, 477-481.
13. Lee, S. W.; Gleason, N. R.; Ssenymanturo, K.; Woodring, J.; Bessler, M.; Whelan, R. L. Colon cancer tumor proliferative index is higher and tumor cell death rate is lower in mice undergoing laparotomy vs. insufflation. Surg. Endosc. 1998, 12, 514.
14. MacDonald, R. G.; Schaffer, B. S. ; Kang, I. J. ; Hong, S. M.; Kim, E. J.; Park, J. H. Growth inhibition and differentiation of the human colon carcinoma cell line, Caco-2, by constitutive expression of insulin-like growth factor binding protein-3. J. Gastroenterol. Hepatol. 1999, 14, 72-78.
15. Milsom, J. W.; Bohm, B.; Hammerhofer, K. A.; Fazio, V.; Steiger, E.; Elson, P. A prospective, randomized trial comparing laparoscopic versus conventional techniques in colorectal cancer surgery: a preliminary report. J. Am. Coll. Surg. 1998, 187, 46-54.
16. Murakami, K.; Matsuura, T.; Hasumura, S.; Nagamori, S.; Yamada, Y. ; Saiki, I. Involvement of insulin-like growth factor binding protein-3 in the retinoic acid receptor-alpha-20 mediated inhibition of hepatocellular carcinoma cell proliferation. Cancer Lett. 2000, 151, 63-70.
17. Pidgeon, G. P.; Harmey, J. H.; Kay, E.; Da Costa, M.; Redmond, H. P.; Bouchier-Hayes, D. J. The role of endotoxin/lipopolysaccharide in surgically induced tumour growth in a murine model of metastatic disease. Br. J. Cancer 1999, 81, 1311-1317.
18. Rajah, R.; Valentinis, B.; Cohen, P. Insulin-like growth 30 factor (IGF)-binding protein-3 induces apoptosis and mediates the effects of transforming growth factor-β1 on programmed cell death through a p53- and IGF-independent mechanism. J. Biol. Chem. 1997, 272, 12181-12188.
19. Robinson, J. R. Sustained and Controlled Release Drug Delivery Systems, ed., Marcel Dekker, Inc., New York, 1978.
20. Shiromizu, A.; Suematsu, T. ; Yamaguchi, K; Shiraishi, N.; Adachi, Y.; Kitano, S. Effect of laparotomy and laparoscopy on the establishment of lung metastasis in a murine model. Surgery 2000, 128, 799-805.
21. Southall, J. C.; Lee, S. W.; Allendorf, J. D.; Bessler, M.; Whelan, R. L. Colon adenocarcinoma and B-16 melanoma grow larger following laparotomy vs. pneumoperitoneum in a murine model. Dis. Colon Rectum 1998, 41, 564-569.
22. Taylor, L. D., et al. Nucl. Acids Res. 1992, 20, 6287-6295.
23. Yu, H.; Rohan, T. Role of the Insulin-like Growth Factor family in cancer development and progression. Review J. Natl. Cancer Inst. 2000, 92, 1472-1489.

### Experimental Details Part 2

1. Kirman I, Cekic V, Poltaratskaia N, Asi Z, Bessler M, Huang E H, Forde K A, Whelan R L. Plasma from patients undergoing major open surgery stimulates in .vitro tumor growth: Lower insulin-like growth factor binding protein 3 levels may, in part, account for this change. Surgery 2002; 132:1 86-92.
2. Cubbage M L, Suwanichkul A, Powell D R. Insulin-like growth factor binding protein-3. organization of the human chromosomal gene and demonstration of promoter activity. J Biol Chem 1990; 265:12642-9.
3. Hong J, Zhang G, Dong F, Rechler M M. Insulin-like growth factor (IGF)-binding protein-3 mutants that do not bind IGF-I or IGF-II stimulate apoptosis in human prostate cancer cells. J Biol Chem 2002; 277:10489-97.
4. Valentinis B, Bhala A, DeAngelis T, Baserga R, Cohen P. The human insulin-like growth factor (IGF) binding protein-3 inhibits the growth of fibroblasts with a targeted disruption of the IGF-I receptor gene. Mol Endocrinol. 1995; 9:361-7.
5. Reeve J G, Kirby L B, Brinkman A. Hughes S A, Schwander J, Bleehen N M. Insulin-like growth-factor-binding protein gene expression and protein production by human tumour cell lines. Int J Cancer 1992; 51:818-21.
6. Grimberg A. P53 and IGFBP-3: apoptosis and cancer protection. Review. Mol Genet Metab 2000; 70:85-98.
7. De Benedetti F, Meazza C, Oliveri M. et al. Effect of IL-6 on IGF binding protein-3: a study in IL-6 transgenic mice and in patients with systemic juvenile idiopathic arthritis. Endocrinology 2001; 142:4818-26.
8. Kirman I, Poltaratskaia N, Cekic V, Forde K, Ansari P, Boulay C, Whelan R L. Depletion of circulating insulin-like growth factor binding protein 3 following open surgery is associated with high IL-6 levels. Dis Colon Rectum 2004, in press.
9. Gill Z P, Perks C M, Newcomb P V, Holly J M. Insulin-like growth factor-binding protein (IGFBP-3) predisposes breast cancer cells to programmed cell death in a non-IGF-dependent manner. J Biol Chem. 1997; 272:25602-7.
10. Boyle B J, Zhao X Y, Cohen P, Feldman D. Insulin-like growth factor binding protein-3 mediates 1 alpha,25-dihydroxyvitamin d(3) growth inhibition in the LNCaP prostate cancer cell line through p21/WAF1. J Urol. 2001; 165:1319-24.
11. Cohen P, Lamson G, Okajima T, Rosenfeld R G. Transfection of the human insulin-like growth factor binding protein-3 gene into Balb/c fibroblasts inhibits cellular growth. Mol Endocrinol 1993; 7:380-6.
12. Ma J, Pollak M N, Giovannucci E. Chan J M, Tao Y, Hennekens C H, Stampfer M J. Prospective study of colorectal cancer risk in men and plasma levels of insulin-like growth factor (IGF)-I and IGF-binding protein-3. J Natl Cancer Inst 1999; 91:620-5.
13. Palmqvist R, Hallmans G, Rinaldi S, Biessy C, Stenling R, Riboli E, Kaaks R. Plasma insulin-like growth factor 1, insulin-like growth factor binding protein 3, and risk of colorectal cancer: a prospective study in northern Sweden. Gut 2002; 50:642-6.
14. Modric T, Silha J V, Shi Z, Gui Y, Suwanichkul A, Durham S K, Powell D R, Murphy L J. Phenotypic manifestations of insulin-like growth factor-binding protein-3 overexpression in transgenic mice. Endocrinology 2001; 142:1958-67.
15. Salahifar H, Baxter R C, Martin J L. Insulin-like growth factor binding protein (IGFBP)-3 protease activity secreted by MCF-7 breast cancer cells: inhibition by IGFs does not require IGF-IGFBP interaction. Endocrinology 1997; 138:1683-90.
16. Manes S, Llorente M, Lacalle R A, Gomez-Mouton C. Kremer L, Mira E, Martinez-A C. The matrix metalloproteinase-9 regulates the insulin-like growth factor-triggered autocrine response in DU-145 carcinoma cells. J Biol Chem 1999; 274:6935-45.
17. Irwin J C, Suen L F, Cheng B H, Martin R, Cannon P, Deal C L, Giudice L C. Human placental trophoblasts secrete a disintegrin metalloproteinase very similar to the insulin-like growth factor binding protein-3 protease in human pregnancy serum. Endocrinology 2000; 141:666-74.
18. Claussen M, Kubler B, Wendland M, Neifer K, Schmidt B, Zapf J, and Braulke T. Proteolysis of insulin-like growth factors (IGF) and IGF binding proteins by cathepsin D. Endocrinology 1997; 138: 3797-3803.
19. Papanikolaou A, Wang Q S, Papanikolaou D, Whiteley H E, Rosenberg D W. Sequential and morphological analyses of aberrant crypt foci formation in mice of differing susceptibility to azoxymethane-induced colon carcinogenesis. Carcinogenesis. 2000; 21:1567-72.
20. Papanikolaou A, Wang Q S, Delker D A, Rosenberg D W. Azoxymethane-induced colon tumors and aberrant crypt foci in mice of different genetic susceptibility. Cancer Lett 1998 14130:29-34.

### Experimental Details Part 3

1. Baxter R C, Martin J L, Tyler M I, Howden M E. (1986) Growth hormone-dependent insulin-like growth factor (IGF) binding protein from human plasma differs from other human IGF binding proteins. Biochem Biophys Res Commun 139:1256-61.
2. Booth B A, Boes M, Dake B L, Knudtson K L, Bar R S. (2002) IGFBP-3 binding to endothelial cells inhibits plasmin and thrombin proteolysis. Am J Physiol Endocrinol Metab 282:E52-8.
3. Booth B A, Boes M, Bar R S. (1996) IGFBP-3 proteolysis by plasmin, thrombin, serum: heparin binding, IGF binding, and structure of fragments. Am J Physiol 271(3 Pt l):E465-70.
4. Boyle B J, Zhao X Y, Cohen P, Feldman D. (2001) Insulin-like growth factor binding protein-3 mediates 1 alpha,25-dihydroxyvitamin d(3) growth inhibition in the LNCaP prostate cancer cell line through p21/WAFl. J Urol 165:1319-24.
5. Cubbage M L, Suwanichkul A, Powell D R. (1990) Insulin-like growth factor binding protein-3. Organization of the human chromosomal gene and demonstration of promoter activity. J Biol Chem 265:12642-9.
6. De Benedetti F, Meazza C, Oliveri M, Pignatti P, Vivarelli M, Alonzi T, Fattori E, Garrone S, Barreca A, Martini A. (2001) Effect of IL-6 on IGF binding protein-3: a study in IL-6 transgenic mice and in patients with systemic juvenile idiopathic arthritis. Endocrinology 142:4818-26.
7. Fowlkes J L, Enghild J J., Suzuki K, Nagase H. (1994) Matrix metalloproteinases degrade insulin-like growth factor-binding protein-3 in dermal fibroblast cultures. J Biol Chem 269:25742-6.
8. Gill Z P, Perks C M, Newcomb P V, Holiy J M. (1997) Insulin-like growth factor-binding protein (IGFBP-3) predisposes breast cancer cells to programmed cell death in a non-IGF-dependent manner. J Biol Chem 272:25602-7.
9. Gronbek H, Thogersen T, Frystyk J, Vilstrup H, Flyvbjerg A, Dahlerup J F. (2002) Low free and total insulinlike growth factor I (IGF-I) and IGF binding protein-3 levels in chronic inflammatory bowel disease: partial normalization during prednisolone treatment. Am J Gastroenterol 97:673-8.
10. Hong J, Zhang G, Dong F, Rechler M M. (2002) Insulin-like growth factor (IGF)-binding protein-3 mutants that do not bind IGF-I or IGF-I1 stimulate apoptosis in human prostate cancer cells. J Biol Chem 277:10489-97.
11. Kirman I, Cekic V, Poltaratskaia N, Asi Z, Bessler M, Huang E H, Forde K A, Whelan R L. (2002) Plasma from patients undergoing major open surgery stimulates in vitro tumor growth: Lower insulin-like growth factor binding protein 3 levels may, in part, account for this change. Surgery 132:186-92.
12. Kirman I, Poltaratskaia N, Cekic V, Forde K, Ansari P, Boulay C, Whelan R L. (2004) Depletion of circulating insulin-like growth factor binding protein 3 following open surgery is associated with high IL-6 levels. Dis Colon Rectum, in press.
13. Lee H Y, Chun K H, Liu B, Wiehle S A, Cristiano R J, Hong W K, Cohen P, Kurie J M. (2002) Insulin-like growth factor binding protein-3 inhibits the growth of non-small cell lung cancer. Cancer Res 62:3530-7.
14. Li L, Yu H, Schumacher F, Casey G, Witte J S. (2003) Relation of serum insulin-like growth factor-I (IGF-I) and IGF binding protein-3 to risk of prostate cancer (United States). Cancer Causes Control 14:721-6.
15. Ma J, Pollak M N, Giovannucci E, Chan J M, tao Y, Hennekens C H, Stampfer M J. (1999) Prospective study of colorectal cancer risk in men and plasma levels of insulin-like growth factor (IGF)-I and IGF-binding protein-3. J Natl Cancer Inst 91:620-5.
16. Manousos O, Souglakos J, Bosetti C, Tzonou A, Chatzidakis V, Trichopoulos D, Adami H O, Mantzoros C. (1999) IGF-I and IGF-II in relation to colorectal cancer. Int J Cancer 83:15-7.
17. Martin J L, Baxter R C. (1986) Insulin-like growth factor-binding protein from human plasma. Purification and characterization. J Biol Chem 261:8754-60.
18. Matsuyama A, Sakai N, Ishigarni M, Hiraoka H, Kashine S, Hirata A, Nakamura T, Yamashita S, Matsuzawa Y. (2003) Matrix metalloproteinases as novel disease markers in Takayasu arteritis. Circulation 108:1469-73.
19. Miyamoto S, Yano K, Sugimoto S, Ishii G, Hasebe T, Endoh Y, Kodama K, Goya M, Chiba T, Ochiai A. (2004) Matrix metalloproteinase-7 facilitates insulin-like growth factor bioavailability through its proteinase activity on insulin-like growth factor binding protein 3. Cancer Res 64:665-71.
20. Renehan A G, Painter J E, Atkin W S, Potten C S, Shalet S M, O'Dwyer S T. (2001) High-risk colorectal adenomas and serum insulin-like growth factors. Br J Surg 88:107-13.
21. Renehan A G, Painter J E, O'Halloran D, Atkin W S, Potten C S, O'Dwyer S T, Shalet S M. (2000) Circulating insulin-like growth factor II and colorectal adenomas. J Clin Endocrinol Metab 85:3402-8.
22. Tchetverikov I, Lard L R, DeGroot J, Verzijl N, TeKoppele J M, Breedveld F C, Huizinga T W, Hanemaaijer R. (2003) Matrix metalloproteinases-3, -8, -9 as markers of disease activity and joint damage progression in early rheumatoid arthritis. Ann Rheum Dis 62:1094-9.
23. Valentinis B, Bhala A, DeAngelis f, Baserga R, Cohen P. (1995) The human insulin-like growth factor (IGF) binding protein-3 inhibits the growth of fibroblasts with a targeted disruption of the IGF-1 receptor gene. Mol Endocrinol 9:361-7.
24. Zhao H, Grossman H B, Spitz M R, Lerner S P, Zhang K, Wu X. (2003) Plasma levels of insulin-like growth factor-1 and binding protein-3, and their association with bladder cancer risk. J Urol 169:714-7.

1. A method for inhibiting the proliferation of cells associated with a tumor in a subject which comprises administering to the subject between about 10 μg/kg and about 100 mg/kg of IGF-BP3, thereby inhibiting proliferation of the cells.

2-15. (canceled)

16. A surgical method which comprises surgically resecting a tumor from a subject and administering to the subject between about 10 μg/kg and about 100 mg/kg of IGF-BP3, so as to inhibit metastasis of any tumor cells released in the subject's blood circulation during the surgical resection of the tumor.

17-33. (canceled)

34. A surgical method which comprises performing a surgical procedure on a subject and administering to the subject between about 10 μg/kg and about 100 mg/kg of IGF-BP3, so as to inhibit proliferation of a tumor cell in the subject.

35-60. (canceled)
